(19) United States
(12) Patent Application Publication
(10) Pub. No.: US 2011/0029004 A1
(43) Pub. Date: Feb. 3, 2011

(54) Tongue cleaning system

(76) Inventor: Stephen Papetti, Monmouth County, NJ (US)

Correspondence Address: ERNEST D. BUFF;ERNEST D. BUFF AND ASSOCIATES, LLC., 231 SOMERVILLE ROAD, BEDMINSTER, NJ, 07921, US

(21) Appl. No.: 12/462,009

(22) Filed: Jul. 28, 2009

(51) Int. Cl. A61B 17/24

(52) U.S. Cl. 606/161

(57) ABSTRACT

A tongue cleaning system uses a disposable glove mitt that has a plurality of mild abrasive strips on the palm facing side of the glove. The user wears the glove mitt and moves the glove up and down on the tongue. The abrasive strips effectively remove food particles which may be decaying while embedded in the crevices of the tongue surface. Also removed are any bio-film that is present. Food taste and oral hygiene are improved, and breath becomes sweet. The tongue cleaning system is disposed after a single use. It may be used anywhere, anytime without the need for water.

### BACKGROUND OF THE INVENTION

1. Field of the Invention

The present invention relates to oral hygiene products; and more particularly to an easy to use sanitary cleaning device for cleaning the tongue at any time or location, without the need for water.

2. Description of the Prior Art

A number of prior art disclosures are related to tongue cleaning devices. These devices do not provide the convenience and functionality of the tongue cleaning system of the subject invention.

U.S. Pat. No. 2,790,982 to Schneider discloses a single use applicator package. This application package applies lotions, oils, or other fluid pastes, which are normally disagreeable to apply. The applicator package is impervious and protects the hand during application of disagreeable fluids contained in the applicator. A single use applicator package dispenses a fluid paste, oil or lotion that is disagreeable to handle. The applicator glove has an impervious layer protecting the hand while a pervious layer such as non-woven cotton or rayon dispenses the fluid paste, oil or lotion. The outer pervious layer may be used to polish after the delivery of the fluid paste, oil or lotion. This device does not have means provided to clean the tongue. The device releases a disagreeable fluid paste, oil or lotion, which may not be applied to a tongue. Moreover, the disagreeable taste of the paste, oil or lotion discourages use, and impairs any cleaning action.

U.S. Pat. No. 4,884,581 to Rescigno discloses an oral hygiene tongue holder. This device is used to manipulate or extend the tongue in order to perform an operation on the tongue or mouth. The device provides non-slip surfaces attached to a user's finger and thumb so that contact with the tongue is made and is manipulated, restrained or extended. This tongue holder has two sides with clasping members that grasp the tongue of a patient by a user, manipulating, restraining or extending the tongue of a patient. The user has the thumb and finger slipped into corresponding arcute loops. The grasping or clamping surface has bristles or other non-slip features to secure the tongue of the patient from both sides. Moreover the grasping surface is not moved along the surface of the tongue. Accordingly, no cleaning action of the tongue is possible; instead, the gasping feature secures the tongue against the griping surface.

U.S. Pat. No. 6,019,773 to Denmark discloses a tongue cleaner. This dental prophylaxis device is for cleansing the tongue and teeth of a user and massaging the gums. It has pair of absorbent core members that have convexo-concave-shapes and have convex surfaces. The core members are is saturated with dental cleansing material. This tongue, teeth cleaner comprises a body that slips over a single finger. The bottom of the tongue, teeth cleaner has a pair of core members wrapped with a terry cloth soaked in antibacterial solution. No disclosure is contained within the '773 patent concerning a mitt that slips over the hand of the user. The tongue, teeth cleaner therein disclosed is, instead, a small pocket, which slips over a finger of the user. Further, the tongue cleaner of the '773 patent does not have a friction surface that can scrape the tongue, but rather relies on a terry cloth that is soaked in an antibacterial composition.

U.S. Pat. Nos. 6,647,549 and 6,721,987 to McDevitt et al discloses a finger glove and a dental wipe respectively. An oral cleaning device fits onto a human finger. The finger glove has two or more sections with a hollow member for the insertion of one or two fingers. One section is non-elastic suited for brushing the teeth, gums or tongue. The second elastic section secures the finger glove on the finger and may be used for polishing the teeth, gums or tongue. The finger glove is not a mit and has abrasive parallel strips adapted for cleaning the tongue.

U.S. Pat. No. 6,808,068 to Abada discloses a portable device for dental hygiene. This is a portable handle less toothbrush with bristles. This portable device does not clean the tongue. The portable device attaches to a finger and does not suggest a mitt that can slipped over a user's hand.

U.S. Pat. No. 6,898,819 to Tanaka et al. discloses a disposable tooth cleaning article. The disposable tooth cleaning article manually cleans tooth surfaces. This disposable tooth cleaning article is wrapped around a finger of the user and is used to clean the tooth. It has flavors for a pleasant feel. This device does not clean the tongue. Further, it is wrapped around a finger and is therefore not a mitt that is attached to a hand.

U.S. Pat. No. 7,020,898 to Pucci et al. discloses a multiuse glove. A hand-worn article includes enclosures for thumb and fingers and a plurality of raised elements, such as bristles, extending in a generally perpendicular direction from the article. The raised elements are attached by heat sealing to the hand enclosure and may be used for a variety of applications, such as tooth brushing, cleaning, polishing, massaging and the like. The article may be constructed as a glove, mitten, an enclosure with an extension, or a hand covering with no receptacles for fingers or thumb. The multiuse glove may be in the form of a fingered glove or a mitt. It has bristled pads attached to selected areas of the glove. These bristled areas are used for brushing cleaning or massaging. There is no suggestion in the '898 patent that the bristles are used for cleaning one's tongue. Moreover, the bristles are only attached as small patches on the tip of the fingers or thumb. Consequently, they do not contact the tongue surface, or otherwise provide a tongue cleaning function. Cleaning of the tongue requires an abrasive surface, not a plurality of bristles.

U.S. Patent Application No. 2002/0198552 to Yavitz discloses an intraoral hygiene device. A method and apparatus is provided for treating breath. This device is a pad having a top surface provided with adhesive to attach the pad to the roof of the mouth. The bottom surface of the pad is at least partially is covered with an abrasive layer. This abrasive layer scrapes the tongue when the device is worn in the mouth. Since the device is attached to the roof of the mouth, it is not a tongue cleaning device that cleans the tongue when cleaning action is needed by the user. Rather, the abrasive pad scrapes the tongue at all times, which can be annoying and may damage tongue tissue.

U.S. Patent Application No. 2003/0037389 to Kuhr discloses a handle-free disposable toothbrush. A tube about the size of an index finger has attached bristles for brushing teeth, gums, and tongue. The tube is additionally provided with one closed end adapted to fit over the finger. The bristles are short, densely placed, and cover at least one-half of the outer surface of the tube. They are saturated with toothpaste, anti-plaque agent, or mouthwash. Optionally, a flexible loop is attached to the open end of the tube. A ¼″ bristle brush disposed on a flexible latex tube is designed to fit over the index finger and is used for brushing teeth, gums and tongue. Such a device does not suggest a mitt that can be placed on a user's hand. Cleaning the tongue requires scraping. Consequently, the soft bristled brush does not do an effective job. The tube only covers the index finger, not the entire hand.

U.S. Patent Application No. 2008/0208228 to Mueller discloses a tongue cleaning device comprising tongue cleaning elements of different materials. This is a handled tongue cleaning device with the bottom surface provided with a plurality of hard and soft polymeric ridges. The opposing upper side of the device has only soft ridges and does not produce any tongue cleaning action. This device appears to be a reusable device, not a disposable device. Consequently, the handed device disclosed by the '228 patent publication does not suggest a mitt with abrasive regions that fits over a user's hand.

U.S. Patent Application No. 2008/0294182 to Gundlapalli et al. discloses disposable oral hygiene instruments, and methods for storing and dispensing same. A thin slender element with two end portions is used to manipulate the device over an extended tongue. The central portion of the device has the tongue scraping ribbon or strip that is coated with a film that includes one or more of a whitening agent, an anti-bacterial agent, an anti-microbial agent and a flavoring agent. The tongue scraping portion is not said to have an abrasive. Rubbing the tongue with a whitening agent, an anti-bacterial agent or a flavoring agent does not effectively clean the tongue or remove food particles from the ridges of the tongue. The device disclosed by the '182 patent publication does not suggest a glove mitt that fits over the hand of a user.

Foreign Patent Application No. WO 2004/080257 to Dobrin et al. discloses a disposable nonwoven cleansing mitt. A disposable nonwoven cleansing mitt is adapted to fit on a user's hand. The disposable nonwoven cleansing mitt releasably contains a personal care composition. This mitt has a front side and a back side joined together at the edges to form a mitt. The front surface carries a releasable personal composition in the form of strips, dots, non-geometric patterns and the like. There is no indication that the personal composition is an abrasive, or that the mitt is used for cleaning the tongue.

Foreign Patent Application No. WO 2005/117740 to Wagner discloses digital wipes and methods of handling. A digital wipe structure, and method for handling the wipe structure, are provided that is particularly useful in forming and handling an oral wipe to clean teeth and gums, especially a young child's teeth and gums. This digital wipe is a sheet of cellulosic or other material soaked in an anti-bacterial solution. It is folded in a specified pattern around the index finger of a user to clean the teeth, gums and tongue of a child. The sheet that is folded is not a mitt that covers the user's hand. It has no abrasive particles to scrape the tongue and thereby provide for effective cleaning.

Foreign Patent Application No. WO 2007/121761 to De Vreese et al. discloses a device for cleaning the oral cavity and a container housing the device. A finger glove has a texturized surface and is optionally provided with a support layer impregnated with detergents and anti-bacterial agents for use in cleaning the oral cavity. The texturized surface is said to break down bio-films. The finger glove of the '761 patent does not fit over the hand of a user. It does not have abrasive portions to scrape the tongue and would likely not clean the tongue thoroughly.

Non-Patent literature “Petrodex Finger Toothbrush Gloves—50 Count Box” at http://www.amazon.com/Petrodex-Finger-Toothbrush-Gloves-count/dp/B001TEM0RC/ref=sr_{—}1_{—}1?ie=UTF8&s=home-garden&qid=1241381376&sr=8-1 is said to have following product features. 1) This 50-count box of Petrodex Finger Toothbrush Gloves helps you clean hard to reach pet teeth much easier than with toothbrushes. 2) Petrodex Finger Toothbrush Gloves present an easy alternative to brushing pets' teeth. Disposable, medical-grade PVC gloves feature bristles on thumb and forefinger. 3) Clean hard-to-reach teeth easier than toothbrushes. Latex-free. 4) Contains 50 average-sized, right-handed gloves. Disposable, medical-grade PVC, gloves are Latex Free. This pet cleaning glove does not suggest a mitt that fits over the hand of the user; and does not have abrasive portions designed to clean a human tongue.

Non-Patent literature “Orymed Finger Brush” at http://www.dentaldepot.com/product/finger_toothbrush_orymed/PRODUCTS_TONGUE_CARE_CLEAN discloses a finger brush with anti-bacterial properties. This antimicrobial cloth wiping device may be rubbed on oral cavity parts to clean the local surfaces. Such a device does not scrape the tongue, and therefore does not expose the pink tongue tissue. It merely kills the upper-most layer of microbes present on the tongue and other oral cavity surfaces.

Notwithstanding the efforts of prior art workers to provide a tongue cleaning system, there remains a need in the art for a tongue cleaning system that is portable and cleans the tongue without need for water.

### SUMMARY OF THE INVENTION

The present invention relates to a tongue cleaning system that comprises a tongue cleaning mitt device and a method of use without water. The tongue cleaning device comprises a mitt having a first cavity appointed to receive fingers of a user. The mitt has a palm surface and a back surface. The palm surface comprises at least one abrasive friction portion appointed to engage with and clean the tongue of said user.

Briefly stated, the invention involves a tongue cleaning system with a tongue cleaning device. More particularly, the tongue cleaning device generally comprises: (i) a mitt having a first cavity appointed to receive fingers of a user; (ii) said mitt having a palm surface and a back surface, wherein said palm surface comprises at least one friction portion appointed to engage with a tongue of said user to clean said tongue. In particular the tongue cleaning device is disposable. It is designed for one time use and can be used by the user anywhere at any time.

### BRIEF DESCRIPTION OF THE DRAWINGS

The invention will be more fully understood and further advantages will become apparent when reference is made to the following detailed description of the preferred embodiments of the invention and the accompanying drawings, in which:

FIG. 1 illustrates a schematic arrangement of the tongue cleaning mitt with the lay out of abrasive containing longitudinal strips oriented generally perpendicular to the fingers; and

FIG. 2 illustrates the second embodiment of the tongue cleaning mitt.

### DETAILED DESCRIPTION OF THE INVENTION

This invention relates to a disposable tongue cleaning device/system with a tongue cleaning mitt that is a disposable and is appointed for single use without need of water. Particularly, the device may be easily used at any location to clean the tongue of the user, thereby improving taste, perception and breath freshness and overall oral hygiene. The user removes the tongue cleaning mitt from a package, slips on the hand and rubs the tongue by back and forth movement, whereby the abrasive strips contained in the mitt engage the surface of the tongue removing or wiping off any food particles and decaying food residue from the interstices of the surface of the tongue.

Briefly stated, the invention involves a tongue cleaning system with a tongue cleaning device. Particularly, the tongue cleaning device generally comprises: (i) a mitt having a first cavity appointed to receive fingers of a user; (ii) said mitt having a palm surface and a back surface, wherein said palm surface comprises at least one friction portion appointed to engage with a tongue of said user to clean said tongue. In particular the tongue cleaning device is disposable. It is designed for one-time use and can be used by the user anywhere, anytime.

A cleansed oral cavity has many advantages. These range from health and hygiene to taste enhancement. In fact, dentists recommend cleaning the tongue daily when brushing one's teeth. However, when one is traveling, hiking or camping it can be difficult to clean one's tongue. For example, picture an individual hiking on the Appalachian Trail where there is no access to home comforts. After a strenuous day of hiking, the individual is thirsty and dinner preparations are in order. The fare may not be fancy but its taste can be markedly improved by cleaning the tongue before and after food consumption. Military personnel and others can further advance hygiene and food taste through use of a tongue cleaning operation. What is more, anyone concerned with dental hygiene or their breath and who, like many today, are constantly “on the run” can benefit by simply cleaning his/her tongue.

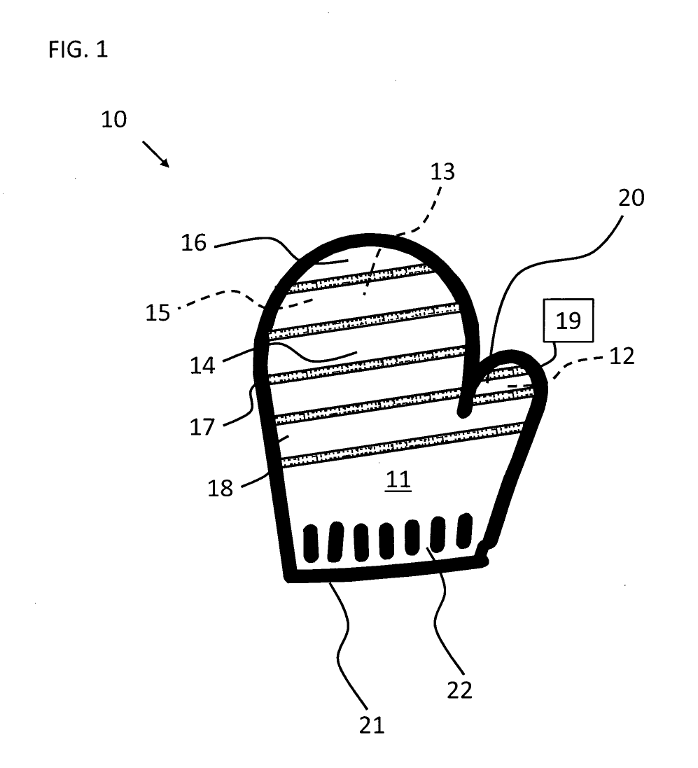

FIG. 1 illustrates a top plan view of an embodiment of the tongue cleaning system, shown generally at 10. In this embodiment the tongue cleaning device is constructed much like a typical mitt. This tongue cleaning device 10 preferably comprises a mitt 11 having a first cavity 13 appointed to receive fingers of a user, accessible through an aperture 21 located on a wrist portion 22 of mitt 11. The mitt 11 may further include a second cavity 12 appointed to receive a thumb of the user. The mitt 11 has a palm surface 14 and a back surface 15. The palm surface 14 comprises at least one friction portion 16 via a slightly roughened surface appointed to engage with a tongue of the user to clean the tongue. In this embodiment the tongue cleaning device is a mitt-shaped cloth article having a slightly roughened exterior surface/friction portion 16. The mitt-shaped cloth article further includes stripes 17 having an enhanced roughness or friction created by mild abrasives, so that friction portion 16 is enhanced. Preferably, these stripes 17 have relatively more abrasiveness than the areas between the stripes, shown as 18. Stripes 19 and areas 20 may also be included on the palm surface 14 of the second cavity 12 so that the user's thumb also facilitates the tongue cleaning procedure.

In operation, a user simply puts his hand inside the mitt 11 and draws the exterior surface or palm surface 14 across the tongue. The mitt 11 has sufficient flexibility to allow the tongue to be grasped and cleaned in a one-hand operation. This facilitates the cleaning operation during such activities as hiking, when trailside conditions make balance an important priority. The exterior surface/palm surface 14 of the mitt 11 can optionally be treated with a mouthwash. The color of the mitt is preferably white. Mitts may be sold as a pack of five individually packaged mitts. They are preferably, disposable; but can be washed, if necessary during use to remove accumulated debris. Thanks to the tongue cleaning device, food flavors are enhanced. The oral cavity is kept clean, and the hiker's hygiene and health are improved.

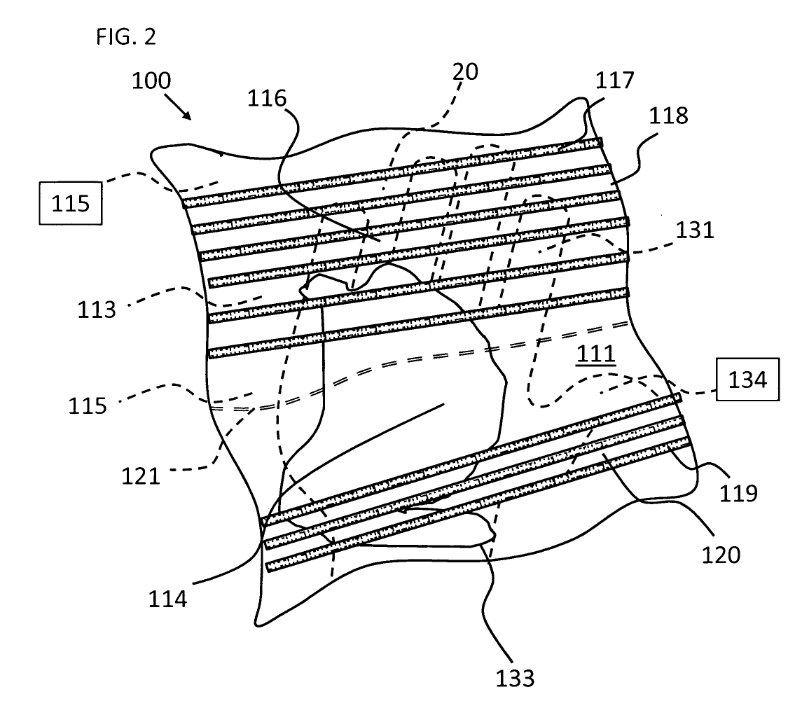

FIG. 2 illustrates a top plan view of a second embodiment of the tongue cleaning device, shown generally at 100. In this embodiment the tongue cleaning device construction comprises a mitt 111 having a first cavity 113 appointed to receive fingers 131 of a user, accessible through an aperture 121 located on a back surface 115 of mitt 111. The mitt 111 herein shown does not include a second cavity for the thumb, but does include material portion 133, which covers the user's thumb 134. The mitt 111 has a palm surface 114 and back surface 115. The palm surface 114 comprises at least one friction portion 116 via a slightly roughened surface appointed to engage with a tongue of the user to clean the tongue. In this embodiment friction portion 116 further includes stripes 117 having an enhanced roughness or friction using mild abrasives so that friction portion 116 is enhanced. Preferably, these stripes 117 have relatively more abrasiveness than the areas between the stripes, shown as 118. Stripes 119 and areas 120 may also be included on the palm surface 114 of the material portion 133 so that the user's thumb 134 also facilitates cleaning of the tongue.

The abrasives used in the strip portions may be based on silica, alumina, walnut shell, wheat husk or other natural products or polymer based abrasives, It is critical that the abrasives have a particle size well below 30 mesh, preferably below 100 mesh but above 300 mesh so that the abrasive strips present on the glove do not damage the top surface of the tongue tissue. Since the strips provided are long with limited width, at any time, only one or two strips contact the tongue, limiting generated friction so that the mitt glove can be easily moved with low level of applied pressure. This limits the overall wear of the top of the tongue tissue and gradually removes the accumulated food debris exposing the pink healthy tongue tissue directly therebeneath.

In its preferred embodiment, the Tongue Cleaning System of the present invention comprises:
i) A glove mitt designed to fit the hand of the user;
ii) said glove having a plurality of narrow long length stripes oriented nearly normal to the finger orientation direction permanently bonded to the glove mitt;
iii) said plurality of long length strips having mild abrasives present in them so that when the glove mitt is rubbed on the tongue by the user, said mitt with long length stripes removes food particles and other accumulated bio-film;
iv) said abrasives having a particle size in the range of 300 mesh to 30 mesh and being composed of inorganic particles including silica, alumina or magnesia, organic natural particles including walnut shells, wheat husk, rice husk, polymeric abrasives or other soft abrasives;
whereby the user can easily translate the glove mitt on the tongue being cleaned with minimal effort and low friction due to the small width of the long length strips in the direction of translation of the glove mitt with minimal surface wear of tongue tissue.

Having thus described the invention in rather full detail, it will be understood that such detail need not be strictly adhered to, but that additional changes and modifications may suggest themselves to one skilled in the art, all falling within the scope of the invention as defined by the subjoined claims.

What is claimed is:

1. A tongue cleaning system comprising:
a. a glove mitt designed to fit the hand of the user;
b. said glove mitt having a plurality of narrow long length stripes permanently bonded thereto and oriented in a direction nearly normal to that of the user's fingers within the glove mitt;
c. said plurality of long length strips having mild abrasives present in them;
d. said mitt with long length stripes and mild abrasives being operative, when the glove mitt is rubbed on the tongue by the user, to remove food particles and other accumulated bio-film;
whereby the user can easily translate the glove mitt on the tongue being cleaned with minimal effort and low friction due to the small width of the long length strips in the direction of translation of the glove mitt with minimal surface wear of tongue tissue.

2. The tongue cleaning system as recited by claim 1, wherein the glove mitt has a finger portion and thumb portion.

3. The tongue cleaning system as recited by claim 1, wherein the glove mitt covers the fingers and the thumb.

4. The tongue cleaning system as recited by claim 1, wherein said abrasives have particles in size ranging from 30 mesh to 300 mesh.

5. The tongue cleaning system as recited by claim 4, wherein said abrasives are particles that are inorganic or organic compounds.

6. The tongue cleaning system as recited by claim 5, wherein said abrasives are inorganic particles comprising silica, alumina, magnesia and combinations thereof.

7. The tongue cleaning system as recited by claim 5, wherein said abrasives are organic particles comprising walnut shells, wheat husk, rice husk, polymeric abrasives, other soft abrasives and combination thereof.

8. A method of using a tongue cleaning system including a glove mitt having a palm surface and an exterior surface, said method comprising the steps of:
a. wearing a disposable glove mitt having a striped abrasive portion facing towards the interior of the palm surface;
b. extending ones tongue from ones mouth;
c. running the tongue mitt up and down along the tongue;
whereby food particles and decaying food residue and bio-film are removed by said strips of abrasives incorporated in said glove mitt's palm facing surface, providing clean taste and good smelling breath without the need for water.

9. A tongue cleaning mitt comprising:
a. a mitt designed to fit a hand of a user, and having a tubular shape;
b. said mitt having an elastic character, fitting closely on the hand;
c. said mitt having an external front surface that is over the palm of the user;
d. said front face of the glove mitt having a plurality of strips that are spaced from each other and oriented perpendicular to the fingers of the user's hand;
e. said strips having a mild abrasive and being operative to remove food debris and bacterial residue when rubbed back and forth over the tongue.

10. A tongue cleaning mitt as recited in claim 9 wherein said strips have abrasive selected from a group comprising silica, alumina, magnesia, walnut shell, wheat husk and polymer based abrasives.

11. A tongue cleaning mitt as recited by claim 10 wherein said abrasives have a particle size in the range of 30 mesh to 300 mesh.

12. A tongue cleaning mitt as recited by claim 10 wherein said abrasives have a particle size in the range of 100 mesh to 300 mesh.
